(19) United States
(12) Patent Application Publication
(10) Pub. No.: US 2007/0119461 A1
(43) Pub. Date: May 31, 2007

(54) Article isolation device and methods

(76) Inventor: Brian Biancucci, Chelsea, MI (US)

Correspondence Address: GOODWIN PROCTER LLP;PATENT ADMINISTRATOR, EXCHANGE PLACE, BOSTON, MA, 02109-2881, US

(21) Appl. No.: 11/600,585

(22) Filed: Nov. 16, 2006

Related U.S. Application Data
(60) Provisional application No. 60/738,190, filed on Nov. 18, 2005.

(51) Int. Cl. A61F 5/37

(52) U.S. Cl. 128/846

(57) ABSTRACT

An article may be isolated by advancing an isolation device disclosed herein to the article, such as a tissue, to be isolated, contacting a terminal edge of the device to a region bordering the article to be isolated, and so applying suction through suction conduits of the device as to seal the article against the terminal edge of the device, thereby isolating the article to be isolated.

### RELATED APPLICATION

This application claims the benefit of U.S. Provisional Patent Application Ser. No. 60/738,190, filed Nov. 18, 2005, the entire disclosure of which is incorporated by reference herein.

### BACKGROUND

Physically and visually accessing organs, tissues and vessels within the body can be important for effective therapy, and such access is often difficult. For example, surgical procedures involving repair of structures within the cardiovascular system often require stopping the heart or otherwise interrupting blood flow in order to create a bloodless field and provide the necessary visual and manual access. The necessity of interrupting normal blood flow adds to the risk and complexity of such a procedure.

Similarly, some drug therapies are designed to treat a specific area of tissue, especially within the cardiovascular system. For example, the direct delivery of such therapeutic agents may minimize undesired side effects from oral or intravenous administration, and/or may maintain extended contact of the therapeutic agent with the target tissue. Direct delivery of such agents is complicated by the difficulty in directing the compound only to the desired area.

It is therefore desirable and beneficial to provide additional and improved methods and apparatus for minimally invasively isolating a target region of tissue from the disruptive effects of pressure and bodily fluids to allow unimpeded access to that region for therapeutic procedures.

### SUMMARY

The present disclosure describes devices and methods for isolating articles from external influences, particularly for isolating biological tissues from contact with external influences such as pressure and bodily fluids.

In one embodiment, an isolation device may include a tube formed by a wall that defines a lumen. The wall may have a proximal portion and a distal portion. The distal portion of the wall may include a distal-most terminal edge that forms a non-planar contour. The distal portion of the wall may also define a plurality of suction conduits that extend to and communicate beyond the distal-most terminal edge.

In another embodiment, an isolation device may include a tube formed by a wall that defines a lumen. The wall may have a proximal portion and a distal portion. The distal portion of the wall may include a distal-most terminal edge and a suction manifold. The distal portion of the wall may also define a plurality of suction conduits that communicate with the suction manifold and extend to and communicate beyond the distal-most terminal edge.

In another embodiment, an isolation device may include a tube formed by a wall that defines a lumen. The wall may have a proximal portion and a distal portion. The distal portion of the wall may include a distal-most terminal edge. The distal portion of the wall may also define a plurality of suction conduits that extend to and communicate beyond the distal-most terminal edge and are lined with metal.

In another embodiment, an isolation device may include a tube formed by a wall that defines a lumen. The wall may have a proximal portion and a distal portion. The distal portion of the wall may include a distal-most terminal edge and may define a plurality of suction conduits that are distributed longitudinally about the distal portion of the wall and alternate with ribbons of stiffener and that extend to and communicate beyond the distal-most terminal edge.

Isolation devices disclosed herein include a device comprising a lumen in communication with an hemispherical isolation tip that includes a plurality of suction conduits. Such a device may include a second set of conduits for delivery of fluids and/or agents to the article.

A collapsible device is also provided, in another embodiment, that includes a lumen in contact with a distal tip of the device, wherein the distal tip of the device includes a plurality of suction conduits, that is biased to open to define an open conical shape. Such suction conduits may have an opening at the base of the conical shape.

A method of isolating an article may include advancing an isolation device disclosed herein to the article, such as a tissue, to be isolated, contacting the terminal edge of the device to a region bordering the article to be isolated, and so applying suction through the suction conduits as to seal the article against the terminal edge of the device, thereby isolating the article to be isolated.

### BRIEF DESCRIPTION OF THE DRAWINGS

FIG. 1 depicts a side view of an isolation device and device tip;

FIG. 2 depicts an end view of the device of FIG. 1;

FIG. 3 depicts a shaped suction conduit;

FIG. 4 depicts a shaped suction conduit;

FIG. 5 depicts a shaped suction conduit;

FIG. 6A depicts two views of a cylindrical isolation tip of an isolation device;

FIG. 6B depicts an oblique (left) and end (right) view of the cylindrical isolation tip inside a vessel;

FIG. 7 depicts an exemplary isolation tip of an isolation device in an expanded configuration (A) and a collapsed configuration (B);

FIG. 8 depicts a side view of an exemplary device insertion into an aorta;

FIG. 9 depicts an end view of a device and the aortic valve;

FIG. 10 depicts a schematic of a device edge suctioned against an aortic leaflet;

FIG. 11 depicts an embodiment of a device tip that includes three collapsible stents in the distal portion;

FIG. 12 depicts an embodiment of a device tip that includes ribbon;

FIG. 13 is a bird's eye view of a device tip that includes webbing;

FIG. 14 is a lateral view of the device tip of FIG. 13;

FIG. 15 is a pictorial depiction of placement of an isolation device before a surgical incision;

FIG. 16 is pictorial depiction of a collapsed device inserted into a ventricle (A) and an expanded device sealing against the ventrical wall (B).

FIG. 17 depicts an overall view of a disclosed device;

FIG. 18 depicts the device tip of the device depicted in FIG. 17;

FIG. 19 is another view of the device tip depicted in FIG. 18;

FIGS. 20 and 21 depict the device of FIG. 17 sealed against an article.

### DETAILED DESCRIPTION

Devices described herein may be used to isolate an article from various outside influences, such as substances and pressures. For example, the devices disclosed herein may be used to isolate a tissue in a living subject from bodily fluids and/or fluid pressures. This may be done by ringing the article to be isolated with a vacuum seal and a wall that is impermeable to the substance or pressure from which the article is to be isolated. The devices disclosed herein provide such a vacuum seal and may also provide a lumen extending to the isolated article, so that manipulations of the article may be performed.

A device may be inserted into the body and isolate a specific area of tissue from blood and blood pressure for the purposes of observing, operating on, or delivering therapeutic agents to the tissue. For example, the disclosed device may isolate a region of a vessel or anatomic chamber for purposes of surgical intervention from the exterior, without significant blood loss. Vacuum may be used to hold the walls of the device against the tissue, while the central portion remains open to the proximal end of the device (for example, open to atmosphere) and can be used for accessing the tissue.

In some embodiments, the device is of appropriate length and diameter for inserting into a peripheral blood vessel and, for example, reaching a distant area of the body (such as the heart). The body of the device contains a lumen which runs through the center of the device, as well as multiple channels which run along and through at least a portion of the wall of the device. The wall channels are oriented around the circumference of the distal end of the device, but may pass through a manifold proximally, allowing a single channel to terminate at the proximal end of the device.

Vacuum is applied at the proximal end, which runs through the wall channels to the tip, and creates a suction seal (preferably a fluid-tight or liquid-tight seal) between the tip wall and a piece of tissue. The central lumen is not exposed to the vacuum, allowing access to the tissue from the proximal end without interference of blood or blood pressure. Blood may be flushed out for visual access with an endoscope, instruments may be passed through the lumen to the tissue, or drugs or other compounds may be delivered to the tissue and allowed to have contact with the isolated tissue for an extended period.

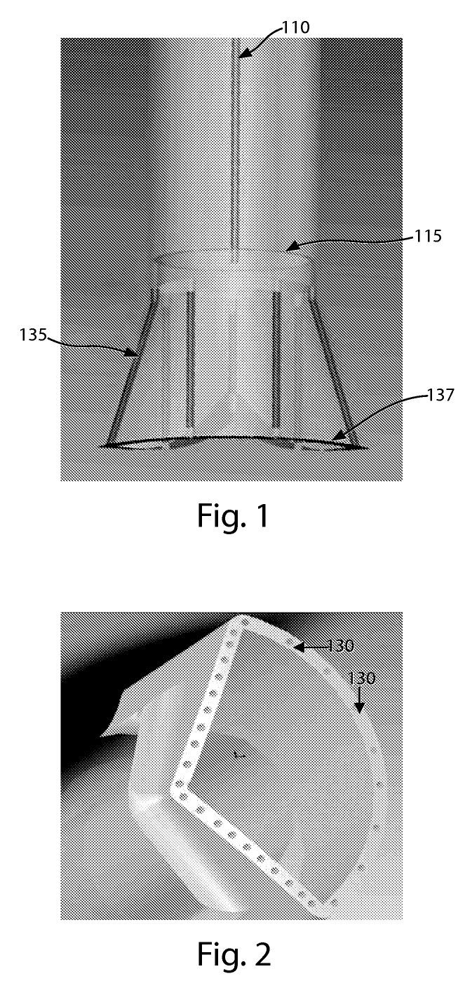

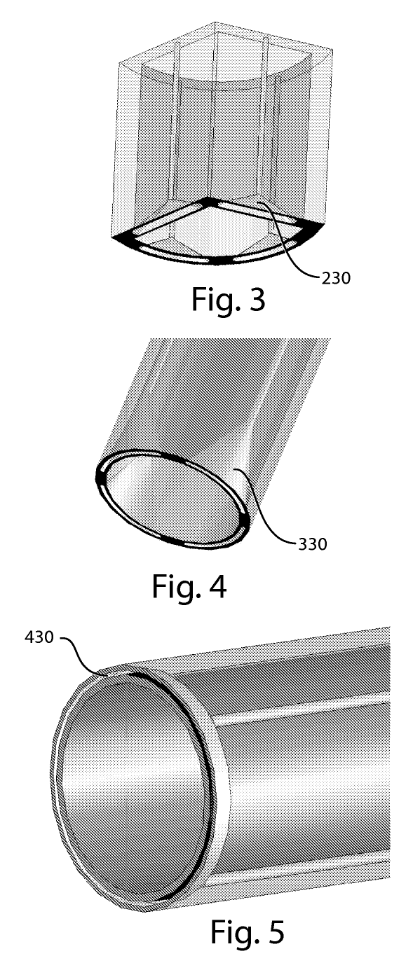

FIGS. 1 and 2 show schematic views of an exemplary embodiment of a portion of an isolation device 100. The device includes a tube formed by a wall that defines a central lumen. The depicted embodiment includes a proximal portion having a single vacuum line 110 running in the wall which communicates with a manifold 115 in the distal portion. The manifold, in turn, communicates with a plurality of vacuum or suction channels or conduits 135 in the distal portion of the device. The suction channels extend to the terminal edge 137 of the distal portion, for example, which terminate as “vacuum holes” 130 and communicate with the space beyond the terminal edge. In some embodiments, the suction conduits may flare at the terminal edge to increase the surface area of suction, as shown in FIGS. 3, 4 and 5. FIGS. 3 and 4 depict different flares shapes 230 and 330, respectively, with differently shaped manifolds. In some embodiments, the suction conduits may not extend to the terminal edge; instead, they terminate short of the terminal edge into an annular space 430 that itself extends to the terminal edge, such as shown in FIG. 5. Such an arrangement may improve seal quality by providing a continuous ring of suction. In an alternative embodiment, a single suction line communicates directly with the annular space.

The device tube may have a wide variety of shapes, as suits the particular manufacturing method or the use to which the device will be put. The tube may have a circular cross-section, non-circular round cross-section, a “sector” cross-section (shown, for example, in FIG. 3), or polygonal cross-sections, such as triangular, square, pentagonal, hexagonal, heptagonal, octagonal, etc. Tubes with polygonal cross-sections may have rounded corners and/or sharp edges.

The depicted terminal edge has a non-planar contour to complement a non-planar article to which the device is to be sealed. In the context of a living organism, a wide variety of articles are contemplated, such as internal and external surfaces of organs such as heart, lung, stomach, small and large intestine, pancreas, bile duct, gallbladder, kidney, ureter, urinary bladder, blood vessels, lymph vessels, etc. Within the heart, articles include valve leaflets, including those of the mitral, aortic, tricuspid, and pulmonary valves, the walls of the left and right atria and ventricles, the interatrial septum, and the interventricular septum. The terminal edge of the device may have a planar or non-planar contour configured to complement the article to be isolated.

In some embodiments, the plurality of suction conduits themselves may extend through the proximal portion of the device wall. A manifold may be provided in the proximal portion of the wall or proximal to the entire device.

The distal end of the device, for example, the terminal edge of the distal end may include a material that allows the terminal edge to deform when pressed against an anatomic structure, e.g. against a cardiac chamber, a vessel, and/or an organ. Such deformation may facilitate isolating the anatomical area of interest. For example, the terminal edge portion may comprise a polymer with a softness that allows effective deformation. Such polymers may include silicone, polyurethane, polyurethane co-polymers, and the like. Suitable polymers may include those with for example a softness of about 20 to about 40 Shore A, as measured by a durometer.

The distal portion of the wall may be collapsible, to facilitate its insertion and removal into, for example, a living organism. The distal portion of the wall may be expanded in a variety of ways. For example, the distal portion may be biased to an expanded orientation, and the removal of a sleeve allows the distal portion to assume an expanded shape. The sheath may be slid back over the distal portion to facilitate removal. The distal portion may be formed at least in part by a material having a shape memory (such as nickel-titanium alloys, generally referred to as “nitinol”) such that exposure to the body temperature of the subject causes the shape-memory material to transition from a collapsed orientation to an expanded orientation. The distal portion may include an inflatable vessel that, when filled with a fluid, causes the distal portion to expand.

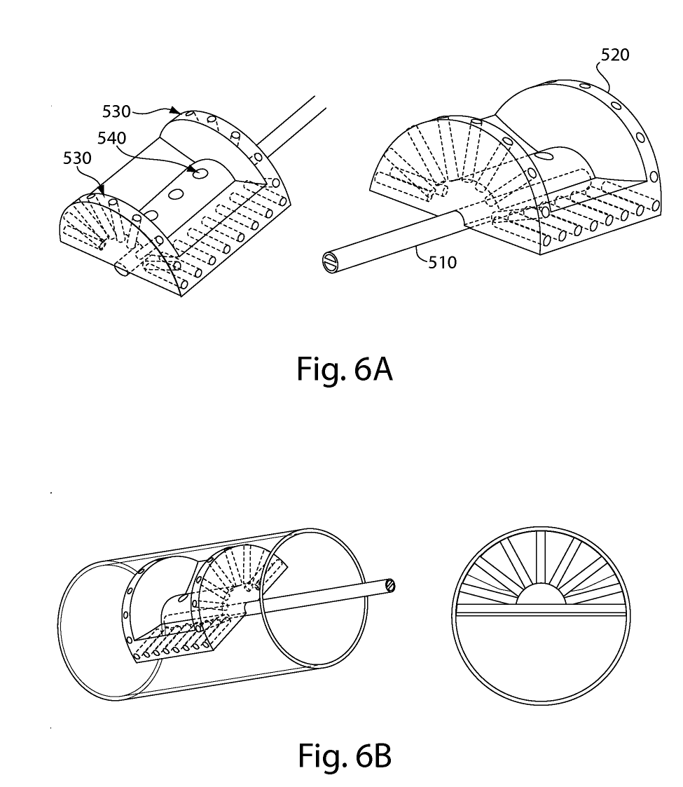

Isolation devices contemplated herein also include those with varying isolation tips, on which a vacuum can be applied. The distal end of the device may be shaped so that it can be applied to a cylindrical vessel, e.g. an artery, as shown in FIG. 6. FIG. 6 depicts a geometric tip configuration 500 that has a hemispherical cross-section and includes an arrangement of vacuum holes 530 on two curved surfaces 520. Such a hemispherical cross-section may facilitate, e.g. half a blood vessel to remain open for blood flow. The vacuum holes of tip 500 are each in communication with one lumen 510, which may be a double lumen catheter, through which vacuum can be applied from the proximal end. The device includes a hollow space including holes 540 which may be in communication with a second lumen of the catheter. Therapeutic agents, for example, can be delivered through this second lumen to the isolated tissue without entering the bloodstream.

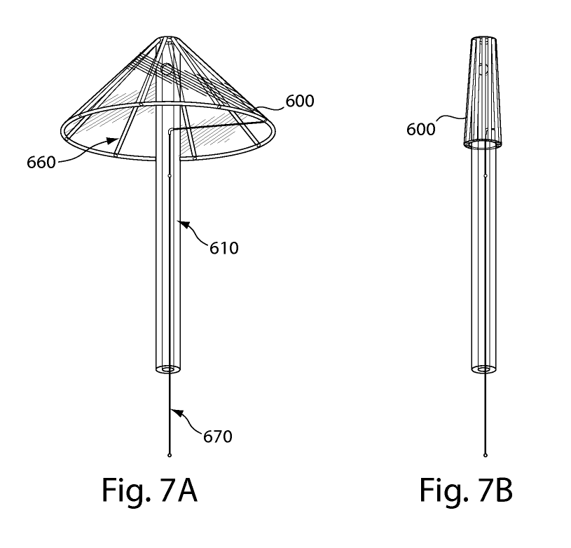

FIG. 7 depicts a device including a collapsible distal tip portion 600, shown in expanded and collapsible forms. Such a device may expand and collapse in a manner similar to an umbrella, and may have an umbrella-like form. Terminal edge 620 includes holes 630 around the perimeter that can be in communication with a lumen 610, which may be a dual lumen. Vacuum may be applied via holes 630 via the proximal end of the device. Distal tip portion 600 may be constructed of a polymer sheet that is embedded with hollow tubes 660 that may act as vacuum channels and may be in contact with holes 630. Hollow tubes 660 can be formed, for example, of a self-expanding material such as nitinol. In the expanded position, tubes 660 may be in the normal, or unstressed, position. Suture or small wire 670 can be secured on the distal tip, e.g. at the terminal edge and directed to the proximal end. Such suture 670 can be used, by e.g. pulling, to cause the tip to collapse and/or releasing such suture 670 can cause the tip to expand.

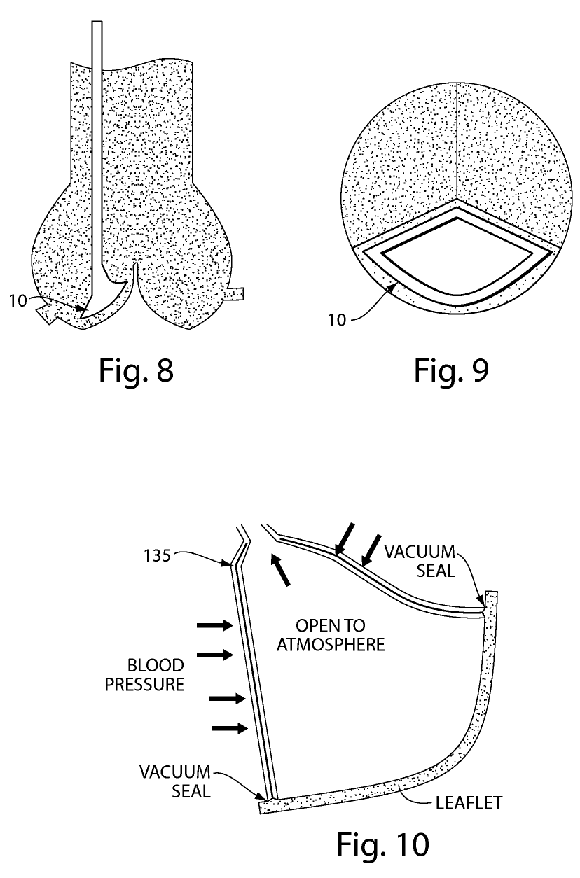

FIGS. 8, 9, and 10 schematically depict an exemplary placement of a device, such as a device depicted in FIGS. 1 and 2, to isolate an aortic valve leaflet, which may be prone to diseases or infections. Several details of the device's structure are omitted for clarity. The device's distal portion has a terminal edge that is contoured to complement the shape of the aortic valve leaflet 10. FIG. 8, 9 and 10 depicts a device has been advanced through a subject's vasculature to reach the aortic valve, pushed from a sheath and allowed to expand. The terminal edge is positioned over the leaflet, and then suction is applied through the suction conduits 135 to seal the terminal edge against the leaflet. The device's central lumen communicates with the isolated leaflet and with the outside, thus allowing observation, manipulation, and/or treatment of the leaflet. Blood and blood pressure, to which the leaflet is normally exposed, are prevented from contacting the leaflet while the device is attached. Such isolation can simplify the desired observation or intervention. For example, a drug may be delivered directly to the leaflet via the catheter. After completing desired intervention, the vacuum pressure can be released, which releases the leaflet.

The distal portion of the disclosed device may include a polymer webbing with small hollow tubes for the vacuum embedded in or adhered to the webbing, such as depicted in FIGS. 13 and 14. The tubes provide rigidity and keep the channel from collapsing during vacuum and provide structure to the otherwise soft tip, allowing a desired geometry to be formed.

Metal tubes inside the tip may be preformed to the desired shape, and then collapsed for insertion through a sheath. When pushed out of the end of the sheath, the tubes will expand, creating the desired tip geometry. The metal tubes may be recessed slightly from the end of the tip so that only the soft material contacts the tissue, thereby increasing the ability to seal against the tissue and minimizing the potential damage to the tissue.

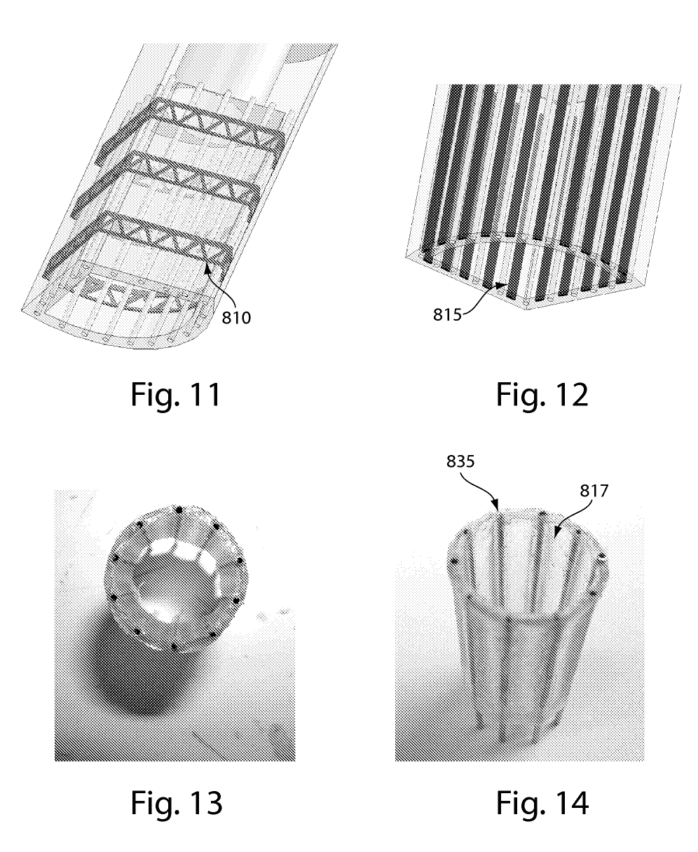

It may be desirable to include stiffening elements in the distal portion of the device to help resist external pressures (such as blood pressure) and/or to resist collapse of the suction conduits due to the suction. To resist external pressures, a stent-like filament may be attached to or embedded in the distal portion of the device wall. The filament may be a collapsible ring or may have a zig-zag or sinusoidal configuration, or other shapes used in stents. The filament may be made of metal, wire, or other material that can be so formed as to have sufficient hoop strength to resist the relevant compressive force. FIG. 11 schematically depicts an embodiment of a device that includes three collapsible stents 810 in the distal portion.

Another way to stiffen the distal portion of the device wall is to interpose suction conduits with ribbons 815 of stiffener, as schematically depicted in FIG. 12. The stiffeners may be made from a wide variety of materials, as described above.

To resist collapse, the suction conduits may be lined with a stiffening material, such as metal, shape-memory metal, nickel-titanium alloy, or plastic, as described above.

The devices described may be put to a variety of uses. In the most basic use, a device is advanced to an article to be isolated, the terminal edge is contacted to the article or to a region surrounding the article, and suction is applied through the suction conduits to seal the article against the terminal edge of the device. In this way, the article is isolated. Such devices can provide for a method of access to such articles.

In some embodiments, the article is a heart valve leaflet, and the terminal edge contour is shaped to complement the leaflet, such as described Example 1.

The disclosed access methods and devices should be useful for a wide variety of procedures to be performed in the isolated space, including fluid withdrawal, drug delivery, diagnostic and therapeutic electrophysiology procedures, pacemaker lead implantation, defibrillator lead placement, transmysocardial revascularization, transmysocardial revascularization with drug delivery, placement of the left ventricular assist devices, placement of the arterial bypass graphs, in situ bypass, i.e., coronary artery-venous fistulae, placement of drug delivery depots, closure of the left arterial appendage, and the like.

For example, tools may be advanced through the lumen of a device to assist in observation, manipulation, and/or treatment of the article. Various endoscopic tools may be used, such as cameras, ultrasound probes, endoscopes, videoscopes, surgical tools (blades, clamps, cautery, staplers, sutures, and the like), tubes for sampling and/or elution of fluids, and a wide variety of other tools. In some embodiments, markers, e.g. radio opaque markers may be placed on the device to allow for detection, e.g. via fluoroscopy.

The article may be treated by contacting it with an agent, and an isolation device can help confine the treatment to the isolated article. The agent may include a bioactive agent, such as an antibiotic, an anti-inflammatory agent, an anti-calcification agent, a thrombolytic agent, a chemotherapeutic agent, nucleic acid, a tissue treatment such as cross-linking agent, a polymer coating, a drug-release polymer (such as a polymer that releases nitric oxide or a nitric oxide precursor), energy, ultraviolet light, broad-spectrum light, radio frequency energy, an ultrasound wave, a cell, and/or a stem cell.

Exemplary agents which may be delivered to the surface of the article, e.g. tissue, may include calcification inhibitors such as osteopontin, osteocalcin, osteonectin, fetuin, albumin and biphosphonates; calcium dissolving agents such as strong acids (e.g. HCl); drugs for the treatment of heart failure, for example anti-arrhythmia drugs (e.g. digoxin, amiodarone, dofetilide, and sotalol), beta-blockers (e.g. carvedilol, propranolol), angiotensin-enzyme converting enzyme (ACE) inhibitors, and calcium channel blockers (e.g. diltiazem, verapamil); anticoagulants or thrombolytic drugs (e.g. aspirin, heparin, coumadin, streptokinase, urokinase, tissue plasminogen activator); drugs for treating vessel wall plaques; crosslinking agents (riboflavin, catechins, glucose); and matrix metalloproteinase (MMP) inhibitors.

In some embodiments, the article includes a heart chamber wall, and a device is advanced through a subject's cardiovascular system to the heart chamber wall. The heart chamber wall may include or have adhered to itself a thrombus, and a thrombolytic drug may be applied to the thrombus through the device's central lumen. The heart chamber wall may define a septal defect, and a tool may be advanced through the lumen of the tube to repair the defect.

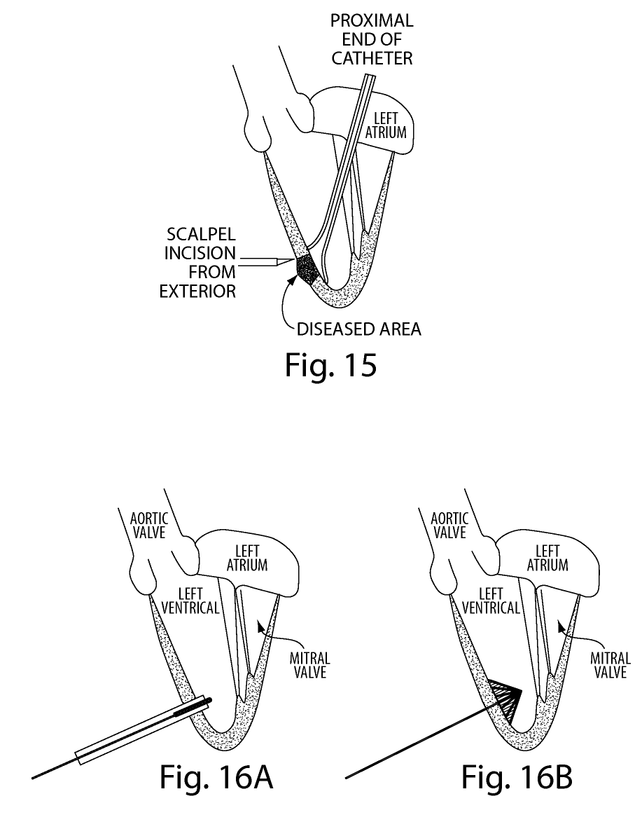

The disclosed devices may isolate a region of a vessel or an anatomic chamber so that blood loss is minimized during a surgical technique. For example, the device may be inserted in a peripheral vessel or a low pressure chamber, for example, the left atrium, and advanced toward the region of interest, e.g. a diseased region, for example left ventricle, as shown in FIG. 15. Once the tip of the device encircles the region of interest, vacuum can be applied, creating a perimeter seal, with the region open to the atmosphere and isolated from blood flow and pressure. Surgical procedures, e.g. excising the diseased tissue or reinforcing weakened or dilated areas, can then be performed from the exterior without significant bleeding or interruption of blood flow, as FIG. 15 illustrates.

Alternatively, the disclosed devices, for example, the exemplary device of FIG. 9, may be inserted in its collapsed configuration into a pressurized chamber, such as the heart or a vessel, by for example, first puncturing the wall of the chamber or vessel from the exterior using a standard introducer with needle. Such an embodiment is depicted in FIG. 16A. The device tip is then a passed through the introducer and into the chamber or vessel, e.g. the left ventricle, and then expanded. Once expanded, the device is pulled back until the edge contacts the inner wall of the chamber or vessel, as shown in FIG. 16B. Vacuum is then applied and the edge seals against the tissue, thereby isolating the area of tissue within the device tip from the surrounding blood and pressure. A second lumen in the catheter body can be used to deliver therapeutic agents to the isolated area of tissue. Alternatively, the isolated area can be operated on from the exterior without blood loss.

In some embodiments, the article to be isolated may form at least part of a fluid-filled chamber, and a tool may be advanced through the lumen of the tube to puncture the chamber.

After using an isolation device to seal against and isolate an area of tissue, a lavage, or fluid rinse, could be performed over the surface of the tissue. Specifically, a rinse solution could be injected into and then withdrawn from the central lumen; the collected solution could then be assayed in order to detect the presence of certain compounds or cells. This information could then be used to diagnose the health of the tissue. For example, measuring nitric oxide end products (i.e., nitrite) would provide an indirect measure of NO production and endothelial cell function. In another example, detecting the presence of inflammatory agents (such as macrophages, etc.) might indicate active inflammation in the tissue. This technique could apply to any diagnostic procedure in which it is desired to sample and measure the local concentration of a particular substance.

In some embodiments, the article is a region of a subject's gastrointestinal tract, and an isolation device may be advanced through the subject's gastrointestinal tract to the region. The region may include an ulcerated portion, and a tool may be advanced, or drug applied, to the ulcer to treat it.

In some embodiments, the article includes a neoplasm, such as a benign or malignant tumor, or a suspicious mass, and a tool may be advanced to sample and/or excise the neoplasm. A chemotherapeutic agent may be applied to the neoplasm through the tube lumen. An ablation tool, such as a radio-frequency energy delivery device, may be advanced to the neoplasm or mass to ablate it.

The invention will now be illustrated by means of the following examples which are given for the purpose of illustration only and without any intention to limit the scope of the present invention.

### EXAMPLE

### Exemplary Device

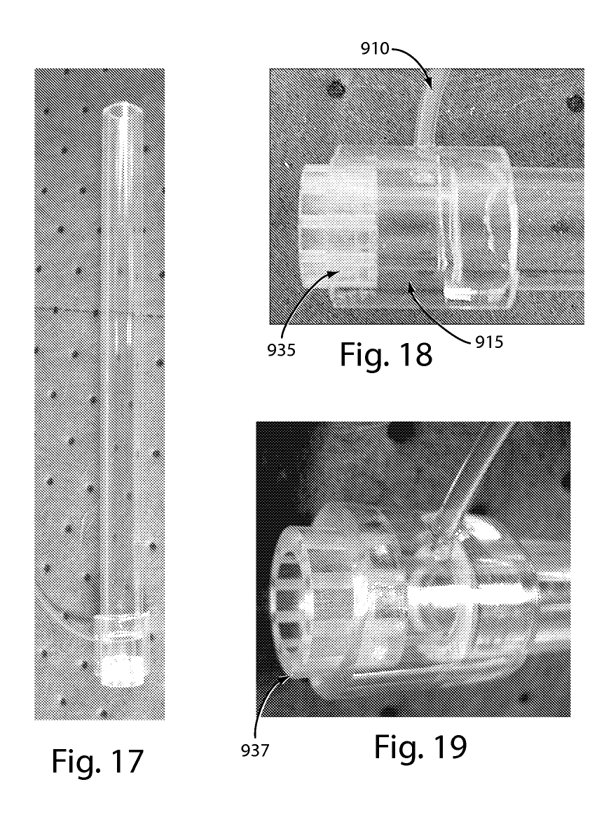

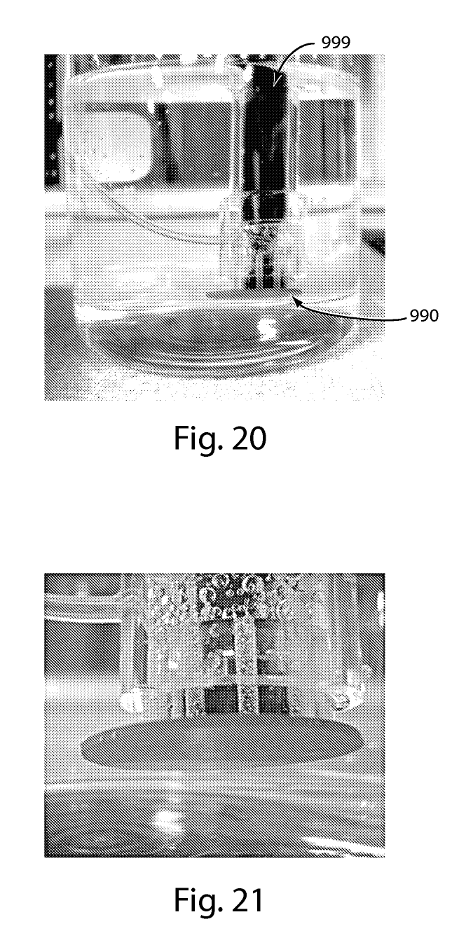

FIGS. 17-21 depict an overall view of an exemplary device as disclosed herein..

The device has a proximal portion and a distal portion. The distal portion has a manifold 915 in communication with a suction line 910 and a plurality of suction conduits 935 arrayed around the distal portion and running longitudinally through the distal wall portion. The suction conduits extend to the terminal edge 937 of the distal portion. In this exemplary embodiment, the terminal edge is planar.

To test this device, an article 990 (in this case, a rubber disk) was placed in a beaker of water. The device was advanced to the disk, and suction was applied through the suction line and suction conduits. The creation of a liquid-tight seal was demonstrated by adding an dyed liquid 999 to the central device lumen. No leakage of dyed liquid was observed.

### INCORPORATION BY REFERENCE

The entire disclosure of each of the documents referred to herein is incorporated by reference for all purposes.

### Equivalents

Although the present invention has been illustrated by means of preferred embodiments thereof, it is understood that the invention intends to cover broad aspects thereof without departing from the spirit and scope of the invention as defined in the appended claims

1. An isolation device comprising a tube formed by a wall that defines a lumen,
wherein:
the wall has a proximal portion and a distal portion;
the distal portion of the wall includes a distal-most terminal edge that forms a non-planar contour; and
the distal portion of the wall defines a plurality of suction conduits that extend to and communicate beyond the distal-most terminal edge.

2. The device of claim 1, wherein the distal-most terminal edge comprises a polymer.

3. The device of claim 2, wherein the polymer has a softness substantially sufficient to allow the terminal edge to deform when pressed against an anatomical surface.

4. The device of claim 1, wherein the contour is shaped to complement an anatomical surface.

5. The device of claim 1, wherein the contour is shaped to complement a substantially cylindrical vessel.

6. The device of claim 1, wherein the suction conduits extend through the proximal portion of the wall.

7. The device of claim 1, wherein the distal portion of the wall further comprises a suction manifold that communicates with the suction conduits.

8. The device of claim 7, wherein the proximal portion of the wall defines a proximal suction conduit in communication with the suction manifold.

9. The device of claim 1, wherein the distal portion of the wall is collapsible.

10. The device of claim 9, wherein the distal portion of the wall is biased to an expanded orientation.

11. The device of claim 9, further comprising a sheath surrounding the tube and holding the distal portion of the wall in a collapsed orientation.

12. The device of claim 11, wherein the sheath is slideably displaceable over the distal portion of the wall.

13. The device of claim 1, wherein said suction conduits are at least partially lined with a stiffening material.

14. The device of claim 13, wherein the suction conduits are lined with metal.

15. The device of claim 13, wherein the suction conduits are lined with a shape-memory material.

16. The device of claim 13, wherein the suction conduits are lined with plastic.

17. The device of claim 1, wherein the plurality of suction conduits are distributed longitudinally about the distal portion of the wall and alternate with ribbons of a stiffener material.

18. The device of claim 1, wherein the distal portion of the wall further includes a filament formed in a zig-zag configuration.

19. The device of claim 1, wherein the distal portion of the wall further includes an inflatable vessel.

20. A method of isolating tissue, comprising:
advancing the device of claim 1 to the tissue to be isolated;
contacting the terminal edge of the device to a region of tissue bordering the tissue to be isolated; and
so applying suction through the suction conduits as to seal the tissue against the terminal edge of the device, thereby isolating the tissue to be isolated.

21. The method of claim 20, further comprising advancing a tool through the lumen of the tube.

22. The method of claim 21, further comprising contacting the tissue to be isolated with an agent through the tube lumen.

23. The method of claim 22, wherein the agent comprises a bioactive agent.

24. The method of claim 20, further comprising advancing a visualization device through the tube lumen and visualizing the isolated tissue.

25. The method of claim 20, wherein the tissue to be isolated is in need of surgical intervention, and wherein the method further comprises advancing a surgical tool through the lumen.

26. The method of claim 20, wherein the tissue to be isolated is in need of surgical intervention, and wherein the method further comprises advancing a surgical tool through external tissue to the isolated tissue.

27. The method of claim 25, wherein the tissue to be isolated comprises a neoplasm or a suspicious mass, and the method further comprising advancing a tool through the lumen of the tube or through external tissue to excise or sample the neoplasm or suspicious mass.
